(19) United States
(12) Patent Application Publication
(10) Pub. No.: US 2011/0318769 A1
(43) Pub. Date: Dec. 29, 2011

(54) METHOD AND COMPOSITION FOR TARGETING VICIOUS CYCLES OF STRESSES AND INFLAMMATORY RESPONSES

(75) Inventor: Shiaw-Der YANG, ROC (TW)

(73) Assignee: NATIONAL TSING HUA UNIVERSITY, ROC (TW)

(21) Appl. No.: 13/166,715

(22) Filed: Jun. 22, 2011

Related U.S. Application Data
(60) Provisional application No. 61/358,183, filed on Jun. 24, 2010.
(60) Provisional application No. 61/366,679, filed on Jul. 22, 2010.

(51) Int. Cl. C12Q 1/48

(52) U.S. Cl. 435/15

(57) ABSTRACT

The present invention provides methods and compositions for targeting the pathologically relevant bipolar actions of these master integrators without affecting the basal physiologically relevant functions. NFA can be used as marker of vicious cycles. Partial inhibition of NFA will be less toxic (in most cases) and more efficient for simultaneous partial inhibitions of all key mediators to terminate this vicious cycles for prevention and treatment of the long-term stresses and chronic inflammation-related diseases without causing any side effects and complications. Aberrant expression of NFA, master stress, and inflammation integrators play a role in orchestrating of vicious cycles. Aspects of the invention provide core technology for simultaneously targeting 12 hallmarks of cancer.

### CROSS-REFERENCE TO RELATED APPLICATIONS

This Non-provisional application claims the benefit of priority to U.S. Provisional Application No. 61/358,183 filed Jun. 24, 2010, and U.S. Provisional Application No. 61/366,679 filed Jul. 22, 2010, the entire contents of which are hereby incorporated by reference.

### FIELD OF THE INVENTION

This application relates to methods of utilizing NFA to determine how NFA correlate with the vicious cycles compounds of OPN, IL-6, and other produced by the body. The application describes a correlation between subject health and NFA.

### BACKGROUND OF THE INVENTION

The “master integrators” of stresses and inflammation responses essential for development and progression of certain illnesses include nuclear factor-κB (NF-κB), interleukin-6 (IL-6), signal transducer and activator of transcription 3 (STAT3) protein, and osteopontin (OPN). The illnesses include: sickness, behavior, aging, obesity, osteoporosis, osteoarthritis, systemic bone loss, pain perception, cognitive impairment, depression, diabetes/insulin resistance, atherosclerosis, fibrosis, kidney stones, restenosis, organ atrophy, asthma, atherosclerosis, cardiovascular disease, chronic obstructive pulmonary disease, inflammatory bowel disease, liver disease, crohn's disease, autoimmune diseases including lupus, multiple sclerosis, and rheumatoid arthritis and nine hallmarks of cancer including pre-metastatic niche formation, aberrant bone marrow niche formation, primary systemic endocrine instigation and development and expansion of leukemia, lymphoma and myeloma (“Certain Illnesses”). Paradoxically, all of these master integrators also have essential, positive roles in many physiological processes. Thus, the therapeutic potential and benefit of targeting these key mediators in diseases prevention and treatments, which is now in clinical trials, has raised the serious complications and pitfalls of such approaches. Although many diseases are generated by dysregulation of these master integrators during long-term stresses and chronic inflammations, it is now clear that complete inhibition of a single factor such as NF-κB is toxic and unlikely to cure the diseases and simultaneous partial down regulation of these master integrators involved in vicious cycles of long-term stresses and inflammations without affecting normal physiological functions is less toxic and more efficient in treating the disease.

Nuclear factor A (NFA) was originally identified as a specific membrane and cytoplasmic activating factor A of ATP·Mg-dependent protein phosphatase but has subsequently been characterized as a multisubstrate/multifunctional proline-directed protein kinase (PDPK). Due to high sequence homology in kinase domain, NFA was regarded as a subtype of GSK 3 (glycogen synthase kinase 3), and renamed as GSK-3α. Although GSK 3/GSK-3β and NFA/GSK-3α have long been regarded as two closely-related signaling molecule, albeit structurally-similar in kinase domain, they are not functionally-equivalent or -redundant as previously conceived in drosophila and rodents when based on human clinical studies as demonstrated in this application. Moreover, intensive study and most attention have focused on GSK-3β but entitled GSK 3 without further specifications in many areas of research and suppression of this kinase may cause tumorigenesis which raises a serious issue concerning how to treat diabetes without causing cancer. As a result, the unique role of NFA has been overlooked for more than a decade. Throughout the application, GSK-3α, will be referred to as NFA, a multisubstrate/multifunctional kinase.

Using NFA as a probe for determining a subject's risk in vicious cycles of chronic inflammations and stresses has never been accomplished. It has been appreciated that a molecule can exist for targeting vicious cycles of chronic inflammations and stresses with less toxic.

### SUMMARY OF THE INVENTION

The present invention provides methods and compositions for targeting the pathologically-relevant bipolar actions of these master integrators without affecting the basal physiologically-relevant functions. NFA can be used as marker of vicious cycles. Partial inhibition of NFA will be less toxic (in most cases) and more efficient for simultaneous partial inhibitions of all key mediators to terminate the vicious cycles for prevention and treatment of the long-term stresses and chronic inflammation-related diseases without causing any side effects and complications. Aberrant expression of NFA, master stress, and inflammation integrators play a role in orchestrating of vicious cycles. Aspects of the invention provide core technology for simultaneously targeting 12 hallmarks of cancer: including stable reprogramming of EMT induction, antiapoptosis, premetastatic niche formation, systemic immunosuppression, aberrant tumor-stroma coevolution and crosstalk, cancer-related inflammation, aberrant sternness, aberrant bone marrow niche formation, bone metastasis and primary systemic endocrine instigation for comprehensive cancer control (FIG. 8).

Current clinical trials target the vicious cycles of chronic stresses and inflammations by complete inhibition of NF-κB, STAT3, IL-6 and/or OPN, but these trials raise serious complications and pitfalls, because such these compounds are important for other tissue function such as immune function and wound healing. In sharp contrast, targeting the more pathologically-relevant signaling NFA should be less toxic and more efficient for simultaneous partial inhibitions of all the key mediators to terminate the vicious cycles. NFA is an orchestrator and integrator of the ubiquitously expressed, OPN-NFκB-IL-6-STAT3-mediated vicious cycles of stresses and inflammations among hematopoietic and mesenchymal stem/progenitor cells, immune/ inflammatory cells and mesenchymal cells. Thus providing a method and composition for targeting such ubiquitously expressed vicious cycles, provides prevention and treatment of aberrant tissue homeostasis, inflammation and stress-related diseases without causing any side effects and complications.

### BRIEF DESCRIPTION OF THE DRAWINGS

FIG. 1 illustrates immunohistochemical staining pattern showing that the poor outcome stroma niches of tumor if associated with NFA (A) concomitantly have substantial upregulations of OPN (B) and IL-6 (C) within inflammatory areas, which is in contrast with the good outcome stroma niches without NFA (D) and with only basal OPN (E) and IL-6 (F).

FIG. 2 depicts immunohistochemical staining pattern showing that the poor outcome stroma niches of hyperplasia tissue if associated with NFA (A and B) concomitantly have substantial upregulations of OPN (C) and IL-6 (D) within inflammatory areas, which is in contrast with the good outcome stroma niches without NFA (E and F) and with only basal OPN (G) and IL-6 (H).

FIG. 3 shows NFA is a novel target for redirecting bad inflammation toward good inflammation and switching from bad niches toward good niches by targeting the vicious cycles of long-term stresses and inflammatory responses.

FIG. 4 illustrates immunohistochemical staining pattern showing that concomitantly upregulations of TGFβ1 (A), TNFα (B), tissue factor (C) and VEGF (D) within NFA-positive (E, F, G and H) poor outcome stromal microenvironments.

FIG. 5 illustrates the existence of NFA in BMC of leukemia patients with progressive disease.

FIG. 6 illustrates Poor outcome tumors if associated with the NFA concomitantly have upregulation of OPN expression within both tumor and stroma.

FIG. 7 illustrates concomitant upregulation of IL-6, TGFβ1, TNFα, tissue factor and VEGF could also be detected within the poor outcome tumor stroma associated with the NFA.

FIG. 8 illustrates the multifaceted role of the NFA as a potential target for viscous cycle and comprehensive cancer control.

### DETAILED DESCRIPTION OF THE INVENTION

For clarity of disclosure, and not by way of limitation, the detailed description of the invention is divided into the subsections that follow.

### Definitions

Unless defined otherwise, all technical and scientific terms used herein have the same meaning as is commonly understood by one of ordinary skill in the art to which this invention belongs. All patents, applications, published applications and other publications, and Genebank Accession numbers referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, the term “NFA” refers to the nuclear proline-directed protein kinase FA also known as glycogen synthase kinase-3α (Woodgett, EMBO J, 1990, 9:2431-8; Yang, Curr Cancer Drug Targets, 2004, 4:591-6). The Genbank Accession numbers for this protein are AAD11986 and AAH27984.

As used herein, “biological sample” refers to any sample from a biologic source, including but not limited to bone marrow, blood, tissue sample, ascites, pleural effusions, body fluids or cell lines.

As used herein, the term “antibody” refers to an isolated or recombinant binding agent that comprises the necessary variable region sequences to specifically bind an antigenic epitope. Therefore, an antibody is any form of antibody or fragment thereof that exhibits the desired biological activity, e.g., binding the specific target antigen. Thus, it is used in the broadest sense and specifically covers monoclonal antibodies (including full length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to scFv, Fab, and Fab2, so long as they exhibit the desired biological activity, e.g., specifically bind NFA.

As used herein, the term “subject” refers to any living organism, preferably the subjects are mammals. Exemplary subjects include, but are not limited to humans.

As used herein, the term “osteopontin (OPN)” refers to intracellular variant of OPN (iOPN) is involved in a number of cellular processes including migration, fusion and motility and it has aberrant expression in vicous cycle .

As used herein, the term “IL-6” refers to one of the most important mediators of fever and of the acute phase response and an interleukin that acts as both a pro-inflammatory and anti-inflammatory cytokine and it has aberrant expression in vicous cycle.

As used herein, the term “stroma niches” refers to the evolutionary results of biological cells, tissue, or organ adaptations to its connective, functionally supportive framework.

### B. Material and Methods:

### Subjects

Clinicopathologic data and the specimens used for immunohistochemical analysis were obtained through a detailed retrospective review of the medical records of patients who had undergone initial tumor resection for cancer at National Taiwan University Hospital, Taipei, Taiwan, between 1987 and 2004. Surgically resected specimens were fixed in 10% formalin and routinely processed for paraffin embedding. Serial sections were stained with hematoxylin and eosin for histological evaluation. Patients were observed until April, 2006. The study was approved by the Institution's Surveillance and Ethics Committee.

Immunohistochemical Analysis: Tissue sections (5 μm) of formalin-fixed, paraffin-embedded tissue containing tumor that showed the maximum extent of tumor cells were dewaxed in xylene and rehydrated in graded concentrations of ethanol. Endogenous peroxidase was blocked with 3% hydrogen peroxide followed by bovine serum albumin blocking for 5 minutes. The slides were next incubated with primary antibody diluted in 0.05 M Tris buffer, pH 7.4, at 4° C. for 16 hours followed by 20-minute incubation at room temperature with super enhancer (Super Sensitive™ Non-Biotin Detection System, [BioGenex, San Ramon, Calif.]), and another 30-minute incubation with polymer-HRP (Super Sensitive™) label. Immunostaining was finally developed with DAB (3-3′ diaminobenzidine tetrahydrochloride). After quenching the enzyme reaction, slides were incubated in DS-enhancer (Zymed, San Francisco, Calif.) at room temperature for five minutes to prevent the interaction between two staining system. Then, slides were incubated with CD34 antibody for one hour at room temperature. After washing, slides were incubated with anti-mouse alkaline phosphatase for 30 minutes at room temperature. BCIP/NBT solution was used for visualisation of the bound antibody. Sections were counterstained with methyl green solution.

### C. Embodiments

### Example 1 and 2

The poor outcome inflammatory stroma niches of tumor if associated with NFA concomitantly have substantial upregulations of OPN and IL-6 within inflammatory areas.

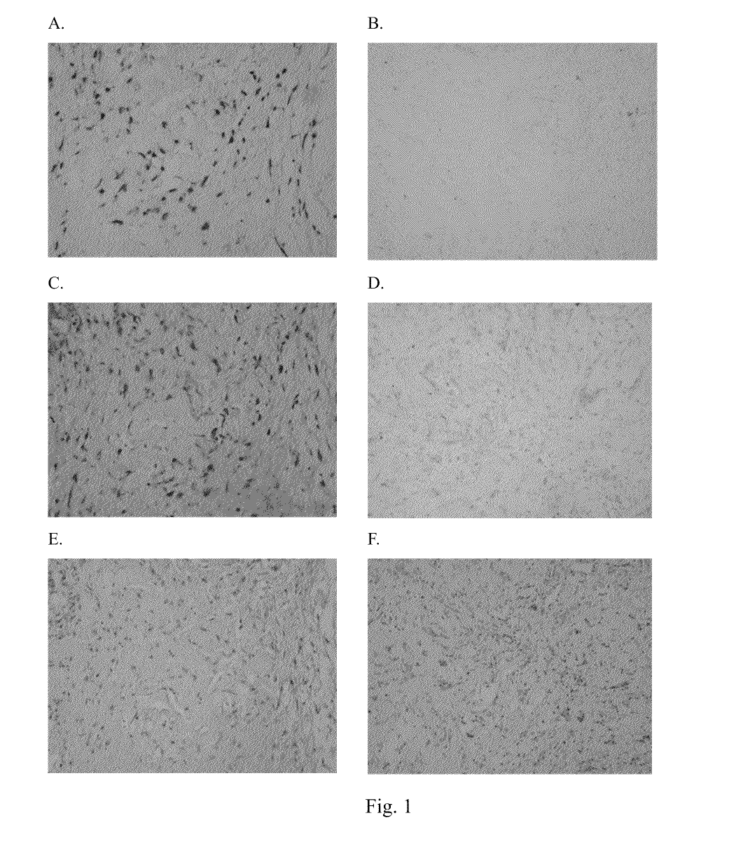

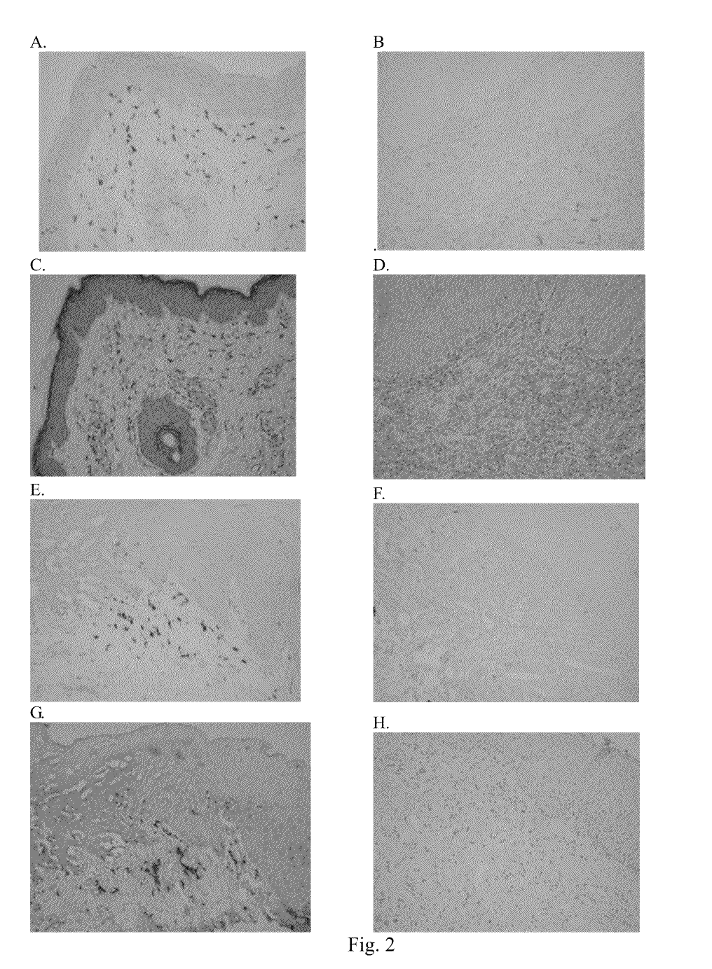

Based on the current paradigm that chronic inflammation, once established, often acquires momentum because of vicious cycles of cytokine and chemokine network. As a result, even a small stimulus raised by a small population of aberrant inflammatory/stromal cells scattered within stromal microenvironments throughout tissues and organs could seed a more systemic inflammatory response. Through using poor outcome and good outcome inflammatory stroma niches of tumor as a model, we showed that the poor outcome stroma niches of breast tumor if associated with NFA (FIG. 1A) concomitantly have substantial upregulations of OPN (FIG. 1B) and IL-6 (FIG. 1C) within inflammatory areas than the good outcome stroma niches without NFA (FIG. 1D) featuring only basal OPN (FIG. 1E) and IL-6 (FIG. 1F). More than 95% of the patients if associated with the presence of NFA and substantial upregulation of OPN and/or IL-6 within inflammatory areas of stroma niches as shown in FIG. 1 had poor outcome even after potentially curative treatments, demonstrating a crucial role of NFA for stromal therapy. Similar observations could also be extended to lung, stomach, colorectum, pancreas, prostate, ovary, kidney and oral cavity. More than 35% of the patients if associated with the presence of NFA and substantial upregulation of OPN and/or IL-6 within inflammatory areas of stroma niches as shown in FIG. 2 had poor outcome even after potentially curative treatments, demonstrating a crucial role of NFA for stromal therapy prior to any carcinoma development.

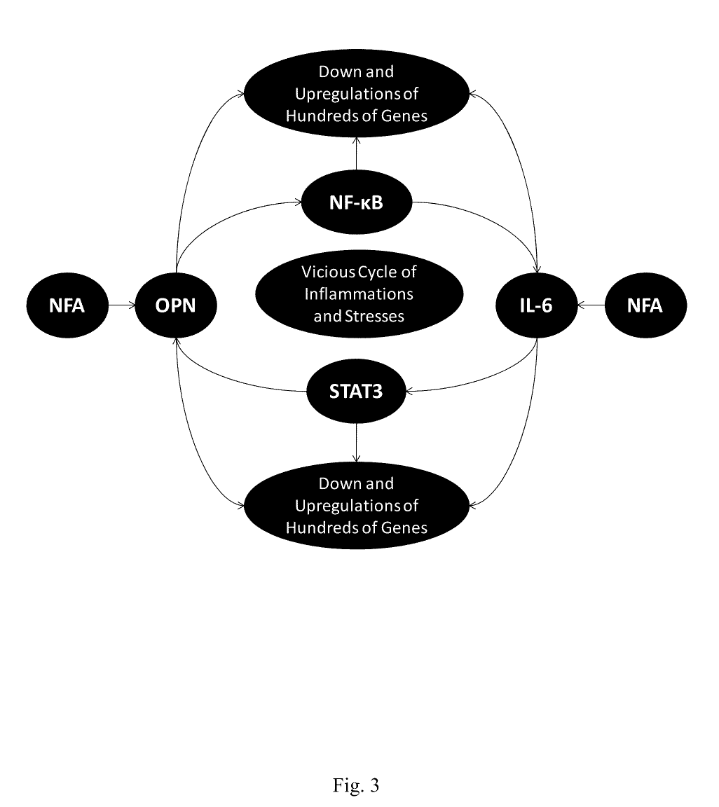

The results as shown in Examples 1 and 2 taken together demonstrate that NFA is a novel target for redirecting bad inflammation toward good inflammation and switching from bad niches toward good niches by targeting the vicious cycles of long-term stresses and inflammatory responses (FIG. 3) for prevention and treatment of Certain Illnesses.

### Example 3

Concomitant upregulations of TGFβ1, TNFα, tissue factor and VEGF within NFA-positive stromal microenvironments.

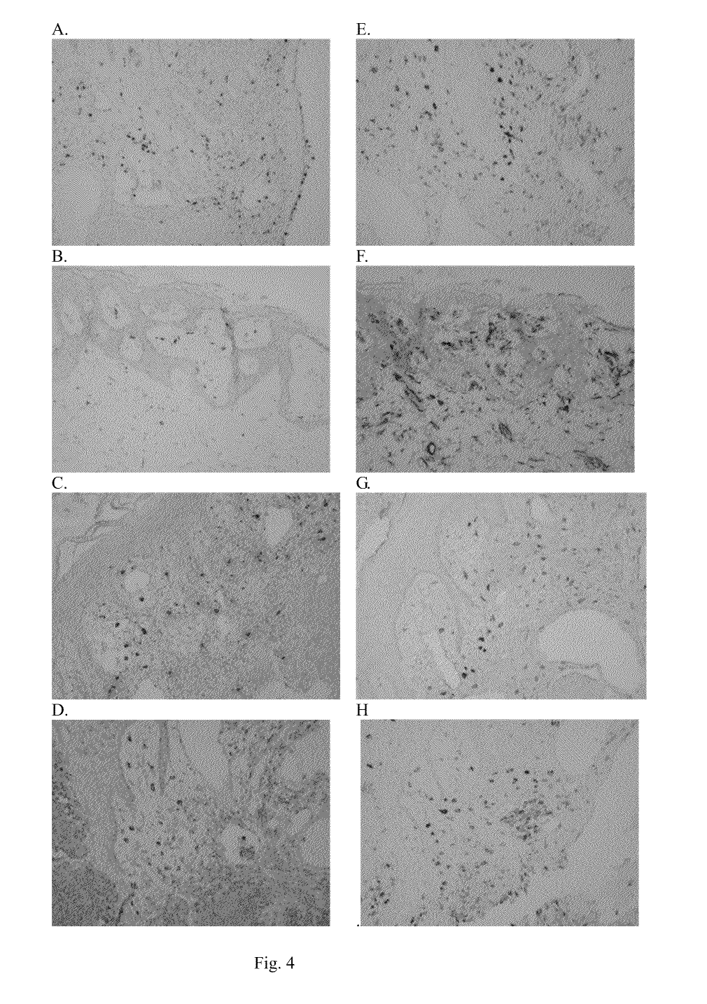

By using similar approaches, concomitant upregulations of TGFβ1 (FIG. 4A), TNFα (FIG. 4B), tissue factor (FIG. 4C) and VEGF (FIG. 4D) could be frequently detected within NFA-positive (FIG. 4E, F, G and H) poor outcome stromal microenvironments as described above. Taken together, the results provide further evidence to support the critical role of NFA in stromal therapy during aberrant tissue renewal, aberrant wound healing and aberrant tissue repair associated with chronic stresses and chronic inflammations.

### Example 4

Expression of NFA in bone marrow cells (BMC) of leukemia patients with progressive diseases.

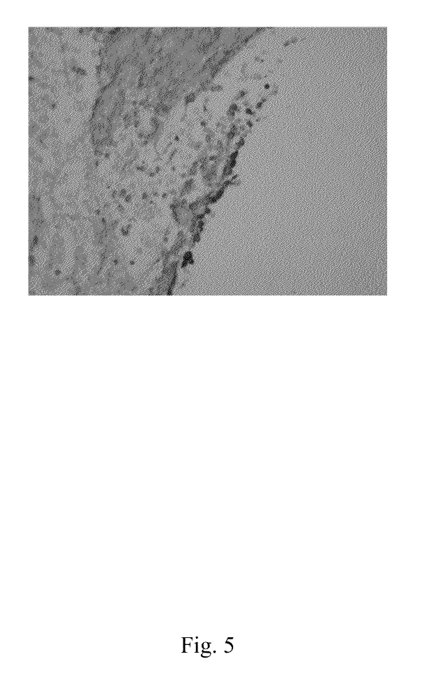

Based on the current paradigm that surveillance of BMC is critically involved in dynamic equilibrium and delicate balance among tissue, bone and vascular remodeling and by using bone marrow as an excellent model, we demonstrate the existence of NFA in BMC of leukemia patients with progressive disease (FIG. 5). Taken together with Examples 1-3, the results further demonstrate the crucial roles of NFA together with OPN and IL-6 in BMC involved in aberrant tissue, bone and vascular remodeling during surveillance and tissue renewal and/or repair during injury, wound healing, chronic stresses and inflammations. Taken together, the the presence of NFA together with OPN, IL-6, TGFβ1, VEGF, TNFα and tissue factor in BMC plays a crucial role in the development and progression of chronic stresses, inflammation and aberrant tissue repair-associated diseases including bone diseases such as systemic bone loss, cardiovascular diseases such as atherosclerosis and inflammation-related diseases and vascular remodeling during surveillance, tissue renewal and wound healing processes. On the other hand, the results also demonstrate the crucial role of NFA in the development of pre-metastatic niche formation, aberrant bone marrow niche formation, primary systemic endocrine instigation and development and expansion of leukemia, lymphoma and myeloma.

Tumors are unhealed and/or overhealing wounds caused by repetitive wounding due to aberrant cell immune response and aberrant tissue repair. The systemic spreading of vicious cycle of stress and inflammation as evidenced within various poor outcome organ and tissue stroma as described above (Examples 1-5) are generated by aberrant expression of NFA. The aberrant expression of NFA indicates elevated levels of master integrators of vicious cycles (FIG. 3). During long-term stress responses and inflammatory responses, aberrant wound healing, aberrant tissue repair, aberrant stem cell niches and aberrant tissue homeostasis formation, can cause the onset or worsening of Certain Illnesses

### Example 5

Poor outcome tumors if associated with the NFA concomitantly have upregulation of osteopontin (OPN) expression

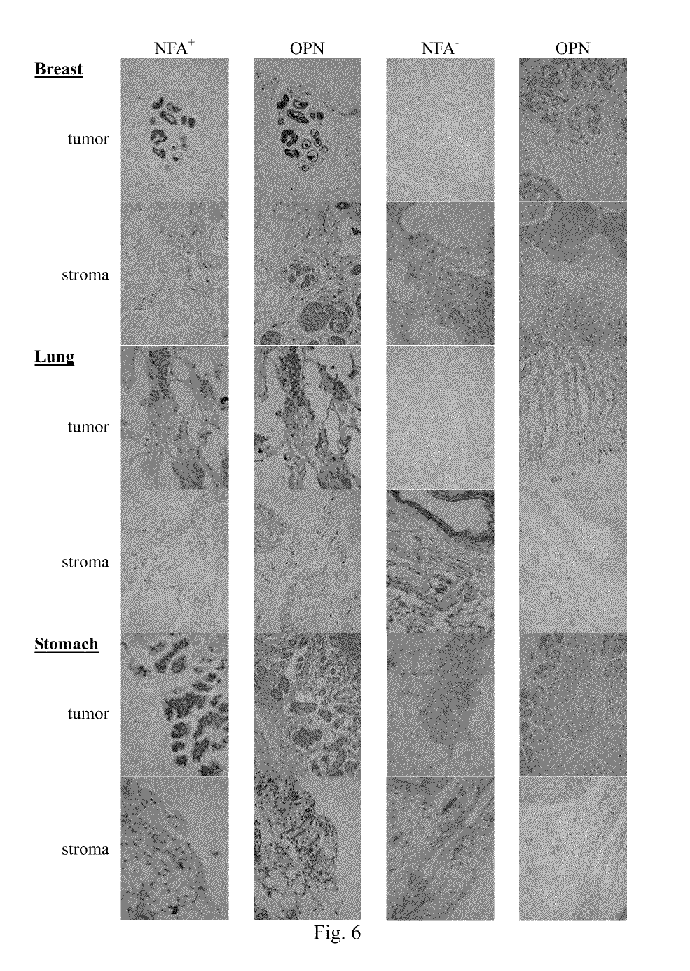

The aggressive breast tumors associated with NFA concomitantly have upregulation of OPN in an exclusive manner within both NFA⁺ (NFA positive) tumor and NFA⁺ stroma, which is in contrast with the NFA⁻ (NFA negative) tumor and NFA⁻ stroma (FIG. 6). Similar observations could also be extended to NFA⁺ poor outcome lung tumor and stroma in contrast with NFA⁻ good outcome tumor and stroma and also in NFA⁺ poor outcome GI tumor and stroma in contrast with NFA⁻ good outcome GI tumor and stroma (FIG. 6). The poor outcome tumors associated with the NFA described above concomitantly have upregulation of OPN expression in an exclusive manner within both breast tumor and stroma, which is in contrast with the NFA− good outcome breast tumor and stroma as indicated. Similar upregulations of OPN expression could also be detected in NFA+ poor outcome lung tumor and stroma in contrast with the NFA− good outcome lung tumor and stroma and also in NFA+ poor outcome GI tumor and stroma in contrast with the NFA− good outcome GI tumor and stroma as indicated. Single staining of NFA and OPN within tumor mass was developed with DAB. The BCIP/NBT solution was used to localize the major population of NFA+vimentin+mesenchymal cells within tumor stroma. Both single and double stainings were performed in duplicates on consecutive tissue sections. The single staining was counterstained with hematoxylin and the double staining was counterstained.

OPN is recognized as an important contributor to tumor progression and metastasis. It is now clear that the multifaceted OPN signaling pathways mediate cancer cell proliferation, antiapoptosis, extracellular matrix invasion, migration, invasion, neovascularization and primary systemic instigation to promote cancer progression and metastasis. The microarray studies revealed the potential of OPN in regulation of 99 genes associated with all the acquired capabilities required for tumor progression and the enabling characteristics of genomic instability. The results provide further evidence to demonstrate that the NFA-tumor-EMT-stroma-BMC-mediated vicious cycle covers primary systemic endocrine instigation, aberrant bone marrow niche formation including bone metastasis and leukemia/lymphoma/myeloma development and expansion and premetastatic niche formation. The results further support a crucial role of NFA in OPN-tumor-EMT-stroma-BMC-OPN coevolutional vicious cycles which are critical for cancer development and progression.

### Example 6

Concomitant upregulations of IL-6, TGFβ1, TNFα, tissue factor and VEGF within NFA-positive tumor-stromal microenvironments.

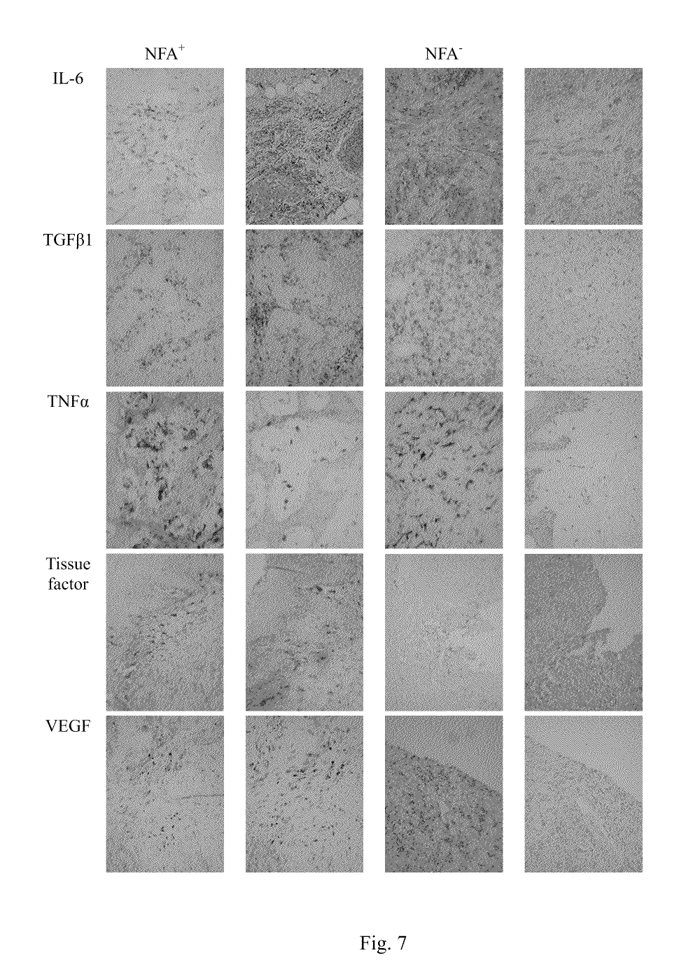

The aggressive tumors associated with the NFA-OPN as described above also concomitantly have upregulations of many key mediators involved in cancer development and progression such as IL-6, TGFβ1, TNFα, tissue factor and VEGF within NFA⁺ poor outcome tumor stromas, which is in contrast with the NFA⁻ good outcome tumor stromas, respectively (FIG. 7). The poor outcome tumors associated with the NFA described above also concomitantly have regulations of IL-6, TGFβ1, TNFα, tissue factor and VEGF within tumor stromas, which is in contrast with the NFA- good outcome tumor stromas, respectively as indicated. Single staining of NFA, IL-6, TGFβ1, TNFα, tissue factor and VEGF were developed with DAB, and counterstained with hematoxylin. The BCIP/NBT solution was used to localize the major population of NFA+vimentin+mesenchymal cells within tumor stromas. Taken together, the results provide further evidence to support the critical role of the NFA for comprehensive cancer control, particularly including bone metastasis.

### Example 7

The NFA is a potential target for comprehensive cancer control.

In a large cohort study, more than 50% of the poor outcome breast cancer patients (44/74) exhibited the NFA as described above. On the other hand, the breast cancer patients if associated with the NFA all failed to have good outcome after the treatment and in a population of 67 good outcome breast cancer patients, no patient exhibited the NFA. Similarly, ˜56% (44/78) of the poor outcome lung cancer patients exhibited the NFA and no false positive case could be detected in a population of 53 good outcome lung cancer patients. Similarly, ˜67% (61/91) of the poor outcome GI cancer patients exhibited the NFA and all had poor outcome after the treatment; no false positive case could be detected in a total of 94 good outcome lung cancer patients after the treatment. Collectively, in a total of 457 cancer patients comprising 214 good outcome and 243 poor outcome cases, more than 60% (149/243) poor outcome patients exhibited the NFA as described in FIGS. 1-4 and all had poor outcome after the treatment. None of the 214 good outcome tumors exhibited the NFA. It is noted that the major population of poor outcome patients were associated with bone metastasis. Taken together, the results demonstrate a crucial role of the NFA predominantly and exclusively in determining the poor clinical outcome of more than 60% of the cancer patients for comprehensive cancer control. The poor outcomes of many cancer patients apparently were predominantly determined by both NFA⁺ BMC and particularly NFA⁺ MTC characteristic of metastatic mesenchymal-like cancer stem cells with multiresistances to immune surveillance, apoptosis, premature senescence, chemotherapy, immunotherapy and current targeted therapy. Thus, NFA represents a newly-described, previously-undiscovered signaling target that plays a pivotal role in the stable maintenance of the mesenchymal state of tumor stem cells involved in cancer progression and metastasis.

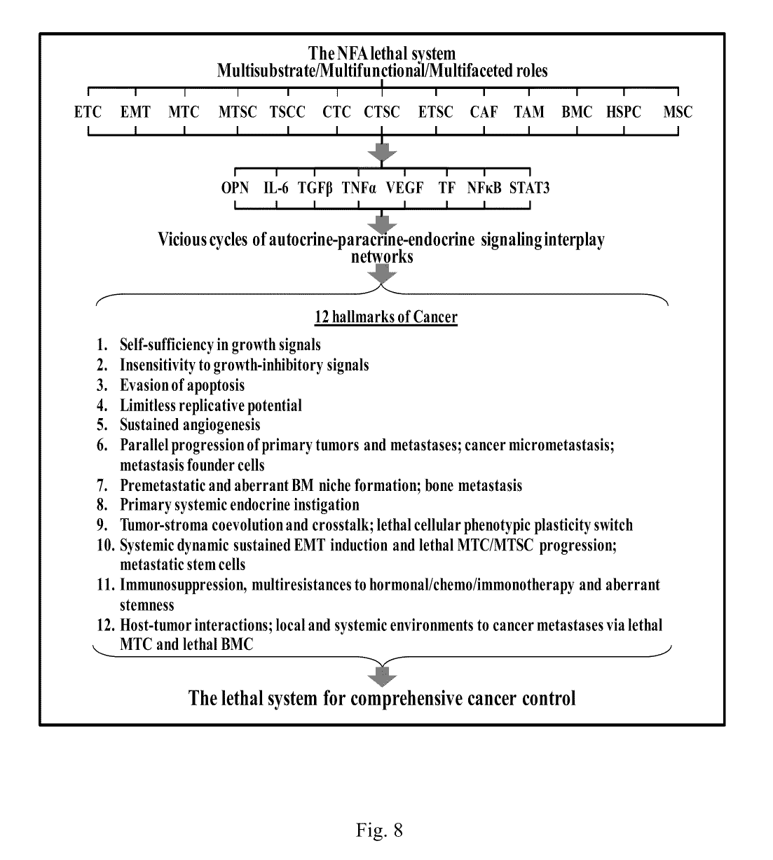

In conclusion, it is now clear that comprehensive cancer control is aided by simultaneous targeting on autocrine, paracrine and systemic endocrine actions of tumor-EMT-stroma-BMC coevolution signaling. Thus, the present invention provides core technology that could simultaneously target 12 hallmarks of cancer including stable reprogramming of EMT induction, antiapoptosis, premetastatic niche formation, systemic immunosuppression, aberrant tumor-stroma coevolution and crosstalk, cancer-related inflammation, aberrant sternness, aberrant bone marrow niche formation, bone metastasis and primary systemic endocrine instigation for comprehensive cancer control (FIG. 8).

What is claimed is:

1. A method for detecting the vicious cycles of inflammations and stresses by performing the steps of:
a. taking a sample from a subject,
b. detecting whether the sample has an NFA marker; and
c. determining the subject has a poor outcome if the sample contains NFA markers.

2. The method of claim 1, wherein said tissue sample is bone marrow, cord blood, peripheral blood, tissue sample, ascites, pleural effusions or body fluids.

3. The method of claim 1, wherein said detection of NFA marker is determined by assessing NFA protein, mRNA, DNA or activity level.

4. A method of determining whether the subject in the vicious cycle of inflammations and stresses by performing the steps of:
a. taking a sample from a subject,
b. detecting whether the sample has an NFA markers; and
c. determining a subject's risk in vicious cycles of chronic inflammations and stresses if the sample contains NFA.

5. The method of claim 4, wherein said sample is bone marrow, cord blood, peripheral blood, tissue sample, ascites, pleural effusions or body fluids.

6. The method of claim 4, wherein said detection of NFA marker is determined by assessing NFA protein, mRNA, DNA or activity level.

7. A diagnostic kit for detecting inflammations and stresses in a sample comprising a method according to claim 1 and a suitable detection means.

8. The method of claim 7, wherein said a suitable detection means comprises one or a plurality of reagents to NFA; and optionally, instructions for use.
